(19) United States
(12) Patent Application Publication
(10) Pub. No.: US 2008/0167477 A1
(43) Pub. Date: Jul. 10, 2008

(54) NOVEL POLYMORPHIC FORMS OF CARVEDILOL DIHYDROGEN PHOSPHATE AND PROCESS FOR PREPARING THE SAME

(75) Inventors: Ramakoteswara Rao JETTI, Hyderabad (IN); Asha Rani GORANTLA, Hyderabad (IN); Om Dutt TYAGI, Hyderabad (IN)

Correspondence Address: WORKMAN NYDEGGER, 60 EAST SOUTH TEMPLE, 1000 EAGLE GATE TOWER, SALT LAKE CITY, UT, 84111, US

(73) Assignee: MATRIX LABORATORIES LIMITED, Secunderabad (IN)

(21) Appl. No.: 11/852,213

(22) Filed: Sep. 7, 2007

(30) Foreign Application Priority Data
Jan. 8, 2007 (IN) 46/CHE/2007
Mar. 9, 2007 (IN) 485/CHE/2007

(51) Int. Cl. C07D 209/88

(52) U.S. Cl. 548/444

(57) ABSTRACT

The present invention provides novel crystalline polymorphic forms and amorphous form of carvedilol dihydrogen phosphate characterized by different solid state techniques. The novel processes for their preparation are also disclosed.

### FIELD OF THE INVENTION

This invention, in general relates to the novel polymorphs of carvedilol dihydrogen phosphate. More particularly, but without restriction to the particular embodiments herein after described in accordance with the best mode of practice, the present invention is directed to crystalline Form A, Form B, Form C, Form D, Form E of carvedilol dihydrogen phosphate dihydrate and amorphous form F of carvedilol dihydrogen phosphate along with the process for the preparing the same.

### BACKGROUND OF THE INVENTION

Carvedilol, 1-(carbazol-4-yloxy-3-[[2-(o-methoxyphenoxy)ethyl]amino]-2-propanol has the formula as given below:

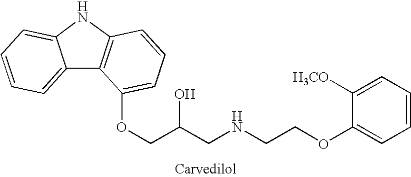

Carvedilol has a chiral center and can exist either as individual stereo-isomers or in racemic form. Racemic carvedilol is the active ingredient of COREG®, which is indicated for the treatment of congestive heart failure and hypertension. Currently the commercially available carvedilol product is a conventional, tablet prescribed as a twice-a-day medication.

U.S. Pat. No. 4,503,067 describes carbazolyl-(4)-oxypropanolamine compounds and its pharmacologically acceptable acid salts thereof. U.S. Pat. No. '067 further discloses the conversion of carbazolyl-(4)-oxypropanolamine compounds into their pharmacologically acceptable acid salts, by reacting with an equivalent amount of an inorganic or organic acid such as hydrochloric acid, hydrobromic acid, phosphoric acid, sulphuric acid, acetic acid, citric acid, maleic acid or benzoic acid in an organic solvent.

PCT publication WO 2004/002419 discloses carvedilol dihydrogen phosphate hemihydrate designated as Form-I, carvedilol dihydrogen phosphate dihydrate designated as Form-II, carvedilol dihydrogen phosphate methanol solvate designated as Form-III, carvedilol dihydrogen phosphate dihydrate designated as Form-IV, carvedilol dihydrogen phosphate designated as Form-V and carvedilol hydrogen phosphate designated as Form-VI, pharmaceutical compositions thereof and methods of using to treat cardiovascular diseases include hypertension, congestive heart failure and angina.

WO 2004/002419 further characterized the claimed crystalline forms of carvedilol phosphate salt through XRD, thermal analysis, FT-Raman spectrums. The processes disclosed for preparation of carvedilol dihydrogen phosphate hemihydrate (Form-I) involves the addition of phosphoric acid to the solution of carvedilol in acetone-water, followed by isolation and drying under vacuum. The process for carvedilol dihydrogen phosphate dihydrate involves the slurrying of the Form-I with acetone/water mixture between 10 and 30° C. for several days, for carvedilol dihydrogen phosphate methanol solvate (Form-III) by slurring Form I in methanol between 10 and 30° C. for several days. WO 2004/002419 further discloses that the carvedilol dihydrogen phosphate displays higher solubility when compared to the free base of carvedilol.

PCT publication WO 2005/051383 discloses several crystalline carvedilol salts selected from mandelate, lactate, maleate, sulfate, glutarate, mesylate, phosphate, citrate, hydrobromide, oxalate, hydrochloride, benzoate as solvates and anhydrous forms. WO 2005/051383 further discloses pharmaceutical compositions containing the above salts, anhydrous forms or solvates thereof and methods of using in the treatment of certain disease in mammals.

Our co-pending application 1028/CHE/2006 discloses sesquihydrate of carvedilol dihydrogen phosphate and process for its production.

In light of the foregoing, novel salt forms of carvedilol with greater aqueous solubility, chemical stability etc. would offer many potential benefits for provision of medicinal products containing the drug carvedilol. In fact the method provides for the above mentioned polymorphs of carvedilol dihydrogen phosphate are not industrially applicable. Therefore, a need exists in the art for identifying an industrially applicable method of producing a stable and easily formulated form of carvedilol dihydrogen phosphate which doesn't require laborious stages and which is sufficiently quick and gentle not to lead to alterations in the final product.

### SUMMARY OF THE INVENTION

It is a principal aspect of the present invention to provide novel crystalline polymorph of the carvedilol dihydrogen phosphate, referred to herein after Form A, Form B, Form C, Form D as a solvates, Form E as an pure dihydrate and Form F as an amorphous form characterized by X-ray powder diffraction pattern, Infrared absorption spectrum, thermo gravimetric analysis (TGA), differential scanning calorimetry (DSC) and/or moisture content.

Furthermore, the present invention is also directed to the process for the preparation of novel crystalline polymorphs of the carvedilol dihydrogen phosphate and amorphous form by using different solvent systems and conditions.

In accordance with one preferred embodiment of the present invention, there is provided a crystalline carvedilol dihydrogen phosphate Form A as a dioxane solvate with a dioxane content of 6-13% supported by thermo gravimetric analysis (TGA) and moisture content of 0.7-1.2% (By KF method).

In accordance with another preferred embodiment of the present invention, there is provided a crystalline carvedilol dihydrogen phosphate Form B.

In accordance with yet another preferred embodiment of the present invention, there is provided a crystalline carvedilol dihydrogen phosphate Form C as a ethanediol solvate with a ethanediol content of 2-6% supported by thermo gravimetric analysis (TGA) and moisture content of 1.0-1.7% (By KF method).

In accordance with yet another embodiment of the present invention, there is provided a crystalline carvedilol dihydrogen phosphate Form D as a ethanediol solvate with a ethanediol content of 6-11% supported by thermo gravimetric analysis (TGA) and moisture content of 3.0% (By KF method).

In accordance with another embodiment of the present invention, there is provided a pure crystalline form E as a dihydrate with a moisture content of 5-7% supported by TGA and 6.3% by KF method.

Furthermore, according to another embodiment of the present invention, there is provided a novel stable Form F of carvedilol dihydrogen phosphate in amorphous state with moisture content of 1-4% (by KF method) and glass transition onset at 71° C. supported by modulated DSC.

Another embodiment provides a process for the preparation of carvedilol dihydrogen phosphate hemihydrate comprising the steps of suspending the carvedilol or its dihydrogen phosphate salt in a solvent and recovered.

### BRIEF DESCRIPTION OF THE DRAWINGS

Further objects of the present invention together with additional features contributing thereto and advantages accruing there from will be apparent from the following description of preferred embodiments of the invention which are shown in the accompanying drawing figures, wherein:

FIG. 1 is the X-ray powder diffraction pattern of Form A of carvedilol dihydrogen phosphate.

FIG. 2 is the DSC of Form A of carvedilol dihydrogen phosphate.

FIG. 3 is the TGA of Form A of carvedilol dihydrogen phosphate.

FIG. 4 is the X-ray powder diffraction pattern of Form B of carvedilol dihydrogen phosphate.

FIG. 5 is the DSC of Form B of carvedilol dihydrogen phosphate.

FIG. 6 is the TGA of Form B of carvedilol dihydrogen phosphate.

FIG. 7 is the X-ray powder diffraction pattern of Form C of carvedilol dihydrogen phosphate.

FIG. 8 is the DSC of Form C of carvedilol dihydrogen phosphate.

FIG. 9 is the TGA of Form C of carvedilol dihydrogen phosphate.

FIG. 10 is the X-ray powder diffraction pattern of Form D of carvedilol dihydrogen phosphate.

FIG. 11 is the DSC of Form D of carvedilol dihydrogen phosphate.

FIG. 12 is the TGA of Form D of carvedilol dihydrogen phosphate.

FIG. 13 is the XRD pattern of carvedilol dihydrogen phosphate dihydrate Form E.

FIG. 14 is the IR of carvedilol dihydrogen phosphate Form E.

FIG. 15 is the DSC of carvedilol dihydrogen phosphate crystalline dihydrate Form E.

FIG. 16 is the TGA of carvedilol dihydrogen phosphate crystalline hydrate Form E.

FIG. 17 is the XRD pattern of carvedilol dihydrogen phosphate amorphous form F.

FIG. 18 is the IR of carvedilol dihydrogen phosphate amorphous Form F.

FIG. 19 is the DSC of carvedilol dihydrogen phosphate amorphous Form F.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention describes the crystalline carvedilol dihydrogen phosphate Form A, Form B, Form C and Form D, which may exists in solvate forms along with the dihydrate Form E and amorphous Form F and are intended to be encompassed with in the scope of the present invention. The said forms are differ from each other in their physical properties, spectral data and method of preparation and characterized by their X-ray powder diffraction patterns, thermo gravimetric analysis (TGA) and/or by infra red absorption spectrum (IR).

Powder X-Ray Diffraction (PXRD)

The said polymorphs of the present invention are characterized by their X-ray powder diffraction pattern. Thus, the X-ray diffraction patterns of said polymorphs of the invention were measured on PANalytical, X'Pert PRO powder diffractometer equipped with goniometer of θ/θ configuration and X'Celerator detector. The Cu-anode X-ray tube was operated at 40 kV and 30 mA. The experiments were conducted over the 2θ range of 2.0°-50.0°, 0.030° step size and 50 seconds step time.

Diffrential Scanning Calorimetry (DSC)

The DSC measurements were carried out on Mettler Toledo 822 Star^{e} and TA Q1000 of TA instruments. The experiments were performed at a heating rate of 10.0° C./min over a temperature range of 30° C.-300° C. purging with nitrogen at a flow rate of 150 ml/min and 50 ml/min. Standard aluminum crucibles covered by lids with three pin holes were used.

DSC Glass Transition

The glass transition temperature (T_{g}) of the amorphous carvedilol dihydrogen phosphate was measured on TA Q1000 of TA instruments with modulated DSC software. The experiments were performed at a heating rate of 3.0° C./min up to a final temperature of 250° with modulation amplitude ±1.0° C., modulation period 80 sec and nitrogen purging at a flow rate of 50 ml/min. Standard aluminum crucibles covered by lids with five pin holes were used.

Thermo Gravimetric Analysis (TGA)

TGA was recorded on out using the instrument Mettler Toledo TGA/SDTA 851^{e} and TA Q5000 of TA instruments. The experiments were performed at a heating rate of 10.0° C./min over a temperature range of 30° C.-300° C. purging with nitrogen at a flow rate of 20 ml/min and 25 ml/min.

Karl-Fisher

Water content was determined on Metrohm Karl-Fisher titrator (Model: 794 Basic Titrino) using pyridine free single solution (Merck, Mumbai) with sample mass between 450 mg to 550 mg.

Infrared Spectroscopy

Fourier transform infrared (FT-IR) spectra were recorded with a Perkin-Elmer spectrum one spectrophotometer. The samples were prepared as 13 mm thickness potassium bromide discs by triturating 1 to 2 mg of sample with 300 mg to 400 mg of KBR by applying pressure of about 1000 lbs/sq inch. Then theses discs were scanned in the spectral range of 4000 to 650 cm⁻¹ with a resolution of 4 cm⁻¹.

Lyophilization

This was carried out using a freeze dryer (Model: Virtis Genesis SQ Freeze Dryer). The virtis Genesis SQ Freeze Dryer operates on the principle of lyophilization, i.e., a process of stabilizing initially wet materials (aqueous solution or suspensions) by freezing them, then subliming the ice while simultaneously desorbing some of the bound moisture (primary drying). Following removal of the ice, desorption may be continued (secondary drying). This process may be carried out under vacuum.

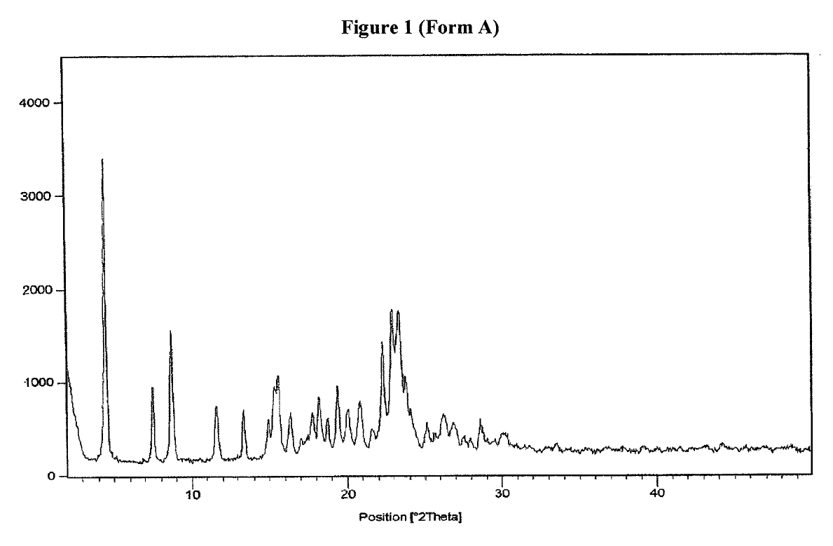

Crystalline carvedilol dihydrogen phosphate Form A is characterized by powder X-ray diffraction pattern as shown in FIG. 1 with peaks at 4.48, 7.48, 8.71, 11.61, 13.36, 14.95, 15.35, 15.60, 16.38, 17.81, 18.20, 18.75, 19.38, 20.05, 20.84, 21.63, 22.31, 22.94, 23.36, 23.79, 25.13, 26.22, 26.84, 28.63 and 30.10±0.2 θ values.

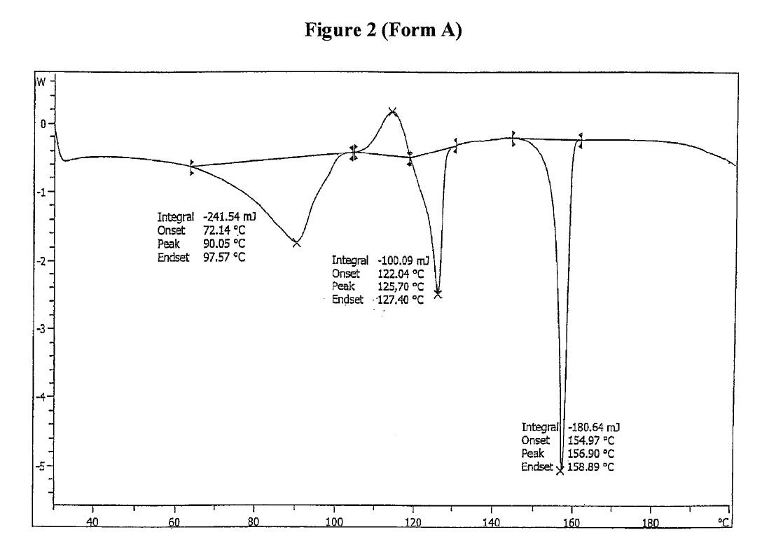

Crystalline carvedilol dihydrogen phosphate Form A is further characterized by DSC with three endothermic peaks first at 90° C. attributed to the loss of the solvent followed by two melting endotherm at 127° C. and 158° C. corresponding melting of the product as shown in FIG. 2. (The water content determined by the Karl-Fisher method is 0.7 to 1.2%)

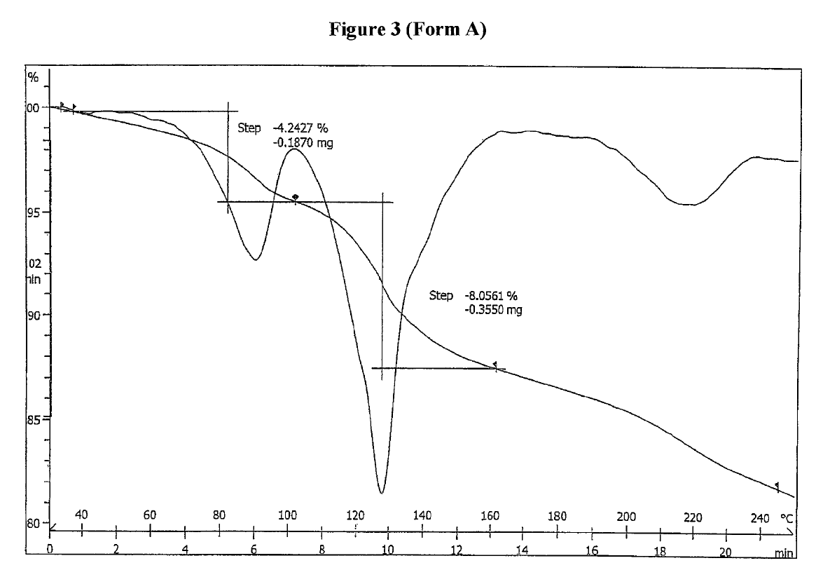

Crystalline carvedilol dihydrogen phosphate Form A is dioxane solvate having between about 6-13% of dioxane content, which is analyzed by its TGA data shown in FIG. 3.

The present invention also provides the process for the preparation of crystalline carvedilol dihydrogen phosphate Form A, which comprises contacting carvedilol dihydrogen phosphate in a solvent, such as 1,4-dioxane, from about ambient temperature to reflux temperature. The obtained solid is filtered under suction followed by vacuum drying. Carvedilol dihydrogen phosphate used herein selected from the group consisting of but not limited to anhydrous or hydrated form.

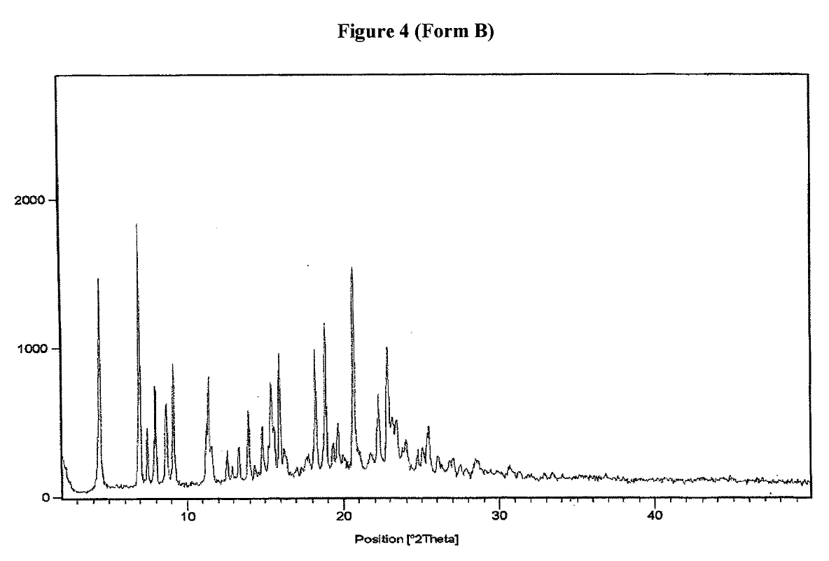

Crystalline carvedilol dihydrogen phosphate Form B is characterized by powder X-ray diffraction pattern as shown in FIG. 4 with peaks at 4.46, 6.98, 7.46, 7.97, 8.68, 9.15, 11.38, 11.60, 12.59, 12.95, 13.33, 13.96, 14.33, 14.84, 15.39, 15.95, 16.22, 17.00, 17.31, 17.72, 18.23, 18.89, 19.35, 19.69, 20.01, 20.68, 20.95, 21.74, 22.27, 22.85, 23.18, 23.37, 23.78, 24.03, 24.75, 25.10, 25.47, 26.07, 26.79, 27.08, 27.48, 28.53, and 30.59±0.2 θ values.

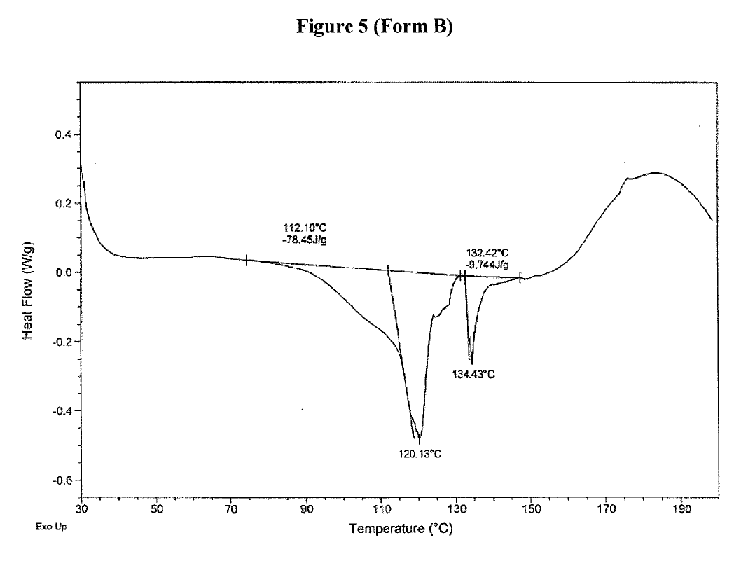

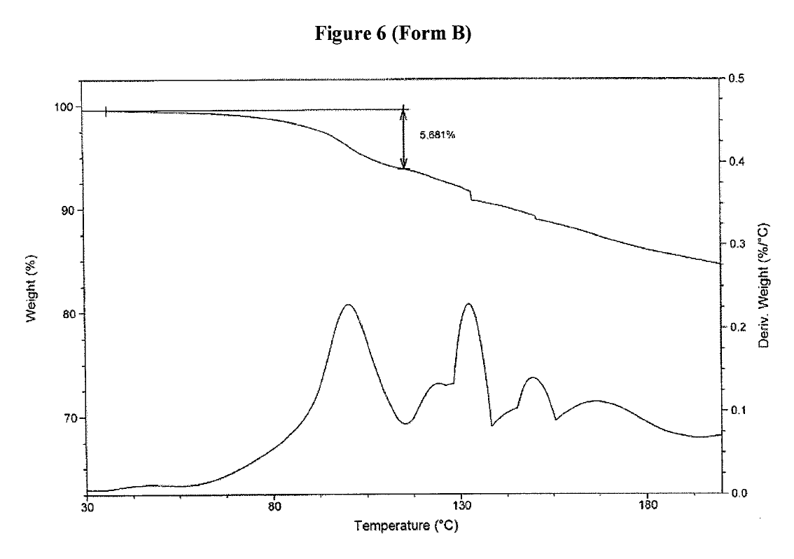

Crystalline carvedilol dihydrogen phosphate Form B is further characterized by DSC with two melting endotherm at 120° C. and 134° C. corresponding melting of the product as shown in FIG. 5 and TGA as depicted in FIG. 6 showing a weight loss of 5.6% due to desolvation.

The present invention also provides the process for the preparation of crystalline carvedilol dihydrogen phosphate Form B, which comprises contacting carvedilol dihydrogen phosphate in a solvent mixture, such as 1,4-dioxane and dimethylformamide (DMF). The obtained solid is filtered and dried under vacuum. Carvedilol dihydrogen phosphate used herein selected from the group consisting of but not limited to anhydrous or hydrated form.

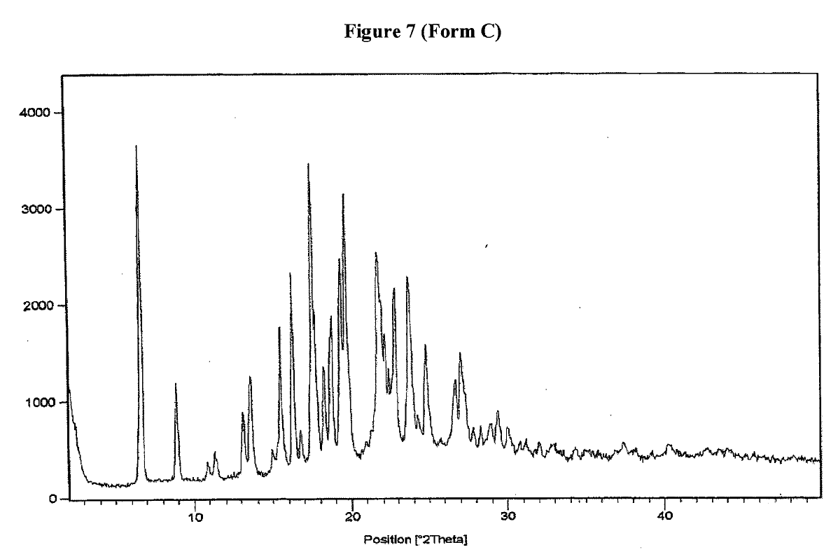

Crystalline carvedilol dihydrogen phosphate Form C characterized by powder X-ray diffraction pattern as shown in FIG. 7 with peaks at 6.60, 8.88, 10.84, 11.30, 13.11, 13.59, 14.93, 15.50, 16.26, 16.76, 17.50, 17.75, 18.28, 18.75, 19.35, 19.68, 21.73, 22.14, 22.80, 23.73, 24.27, 24.79, 25.71, 26.66, 27.03, 27.78 28.26, 28.87, 29.39, 30.03, 30.75, 31.15, 32.01, 32.86, 34.29 and 37.34±0.2 θ values.

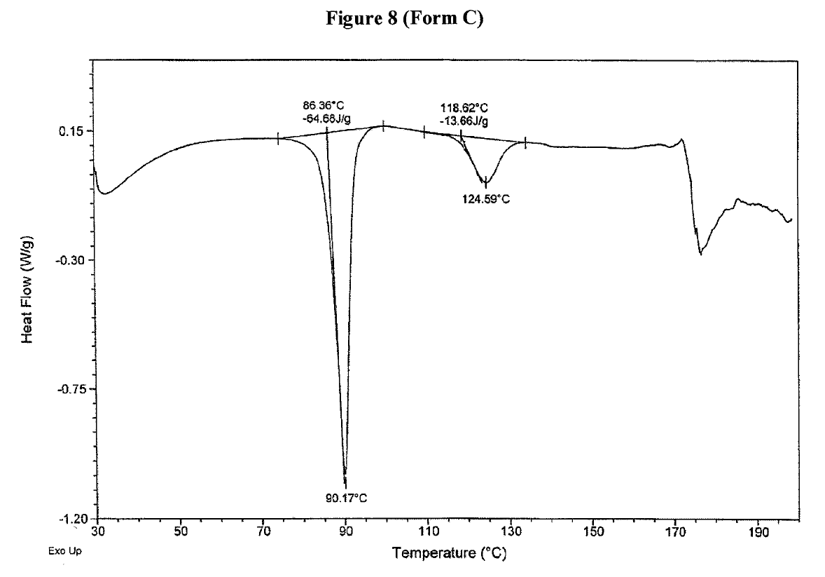

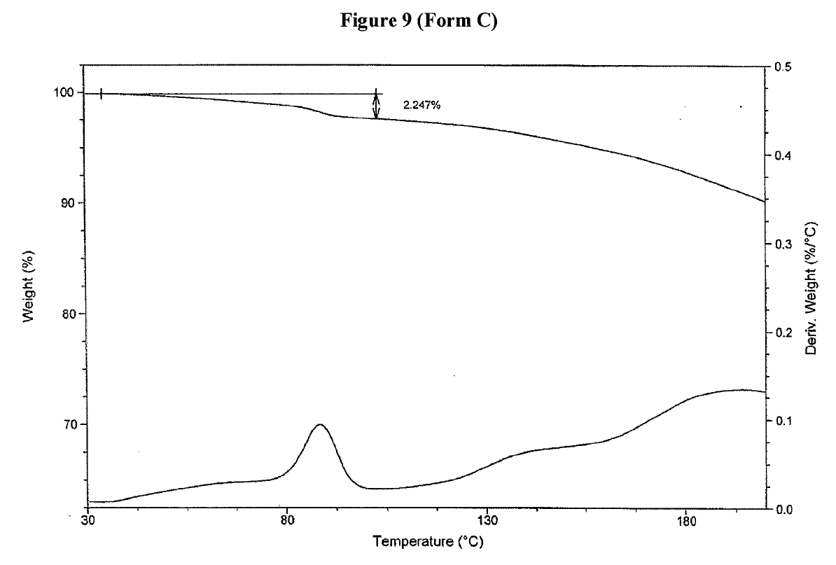

Carvedilol dihydrogen phosphate Form C, is further characterized by the DSC (FIG. 8), it shows two melting endothermic peaks at 90° C. and 125° C. Crystalline carvedilol dihydrogen phosphate Form C is ethanediol solvate having 2-6% of ethanediol content, which is analyzed by its TGA data as shown in FIG. 9. (The water content determined by the Karl-Fisher method is 1.0 to 1.7%)

The present invention also provides a process for the preparation of crystalline carvedilol dihydrogen phosphate Form C, which comprises contacting carvedilol dihydrogen phosphate in a solvent such as 1,2-ethanediol, using the method of slow evaporation. The method of slow evaporation may be either by saturating the carvedilol dihydrogen phosphate in the solvent from about ambient temperature to reflux temperature, followed by cooling the solution to room temperature and then allowing the mixture to stand for an extended period of time (for example, overnight to few days), followed by isolating the product and dried under vacuum. carvedilol dihydrogen phosphate used herein selected from the group consisting of but not limited to anhydrous or hydrated form.

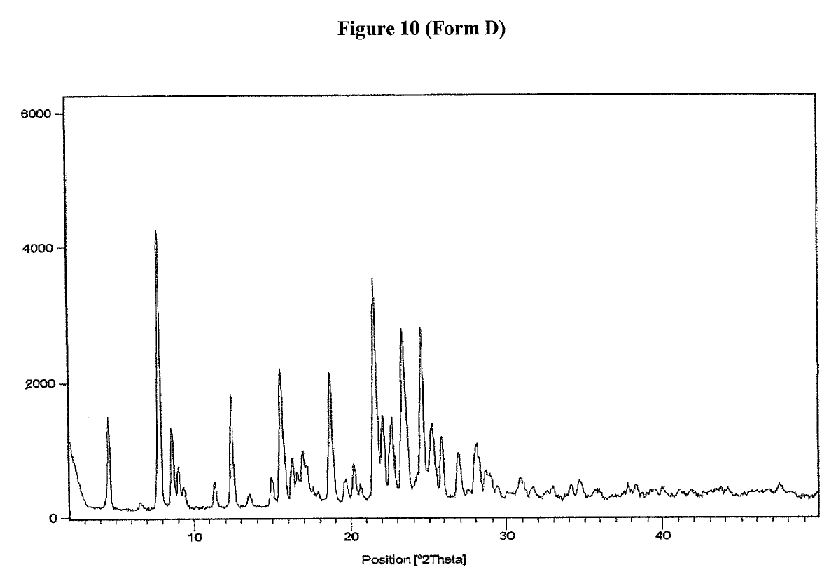

Crystalline carvedilol dihydrogen phosphate Form D characterized by powder X-ray diffraction pattern as shown in FIG. 10 with peaks at 4.54, 6.63, 7.80, 8.63, 9.03, 9.36, 11.37, 12.43, 13.51, 14.95, 15.54, 16.28, 16.95, 18.69, 19.68, 20.24, 20.63, 21.55, 22.08, 22.67, 23.35, 24.54, 25.2, 25.84, 26.90, 28.05, 28.68, 30.92, 31.66, 33.00, 34.13, 34.70, 37.74, 38.28 and 47.47±0.2 θ values.

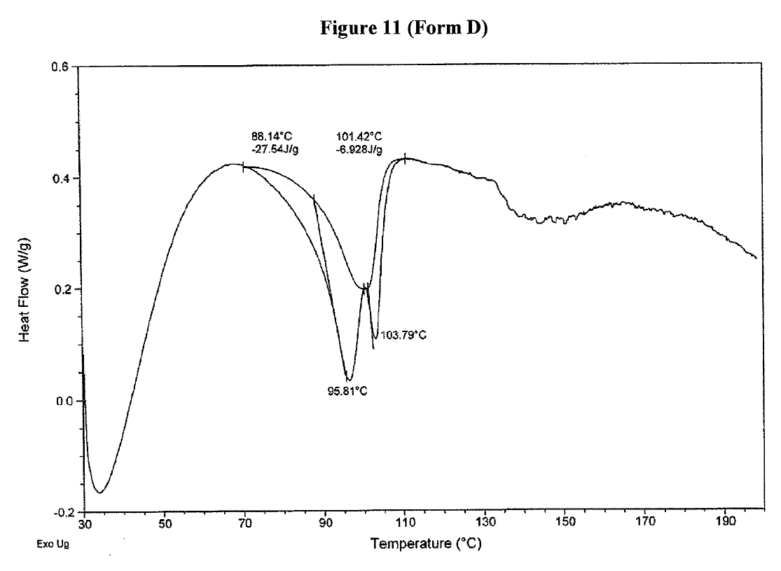

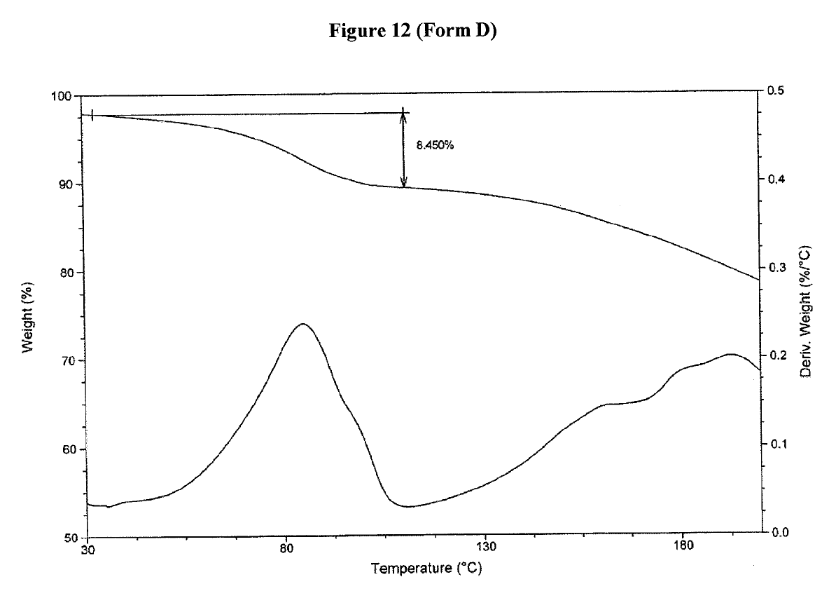

Carvedilol dihydrogen phosphate Form D, is further characterized by the DSC (FIG. 11), it shows two melting endothermic peaks at about 96° C. and 104° C. Crystalline carvedilol dihydrogen phosphate Form D is ethanediol solvate having 6-11%, which is analyzed by its TGA data as shown in FIG. 12. (The water content determined by the Karl-Fisher method is 3.0%).

The present invention also provides a process for the preparation of crystalline carvedilol dihydrogen phosphate Form D, which comprises slow evaporation of saturated solution of carvedilol dihydrogen phosphate from a solvent, such as 1,2-ethanediol, at ambient temperature for several days. Carvedilol dihydrogen phosphate used herein selected from the group consisting of but not limited to anhydrous or hydrated form.

Crystalline carvedilol dihydrogen phosphate Form D can also be prepared by seeding the saturated solution of carvedilol dihydrogen phosphate in ethanediol. Carvedilol dihydrogen phosphate used herein selected from the group consisting of but not limited to anhydrous or hydrated form.

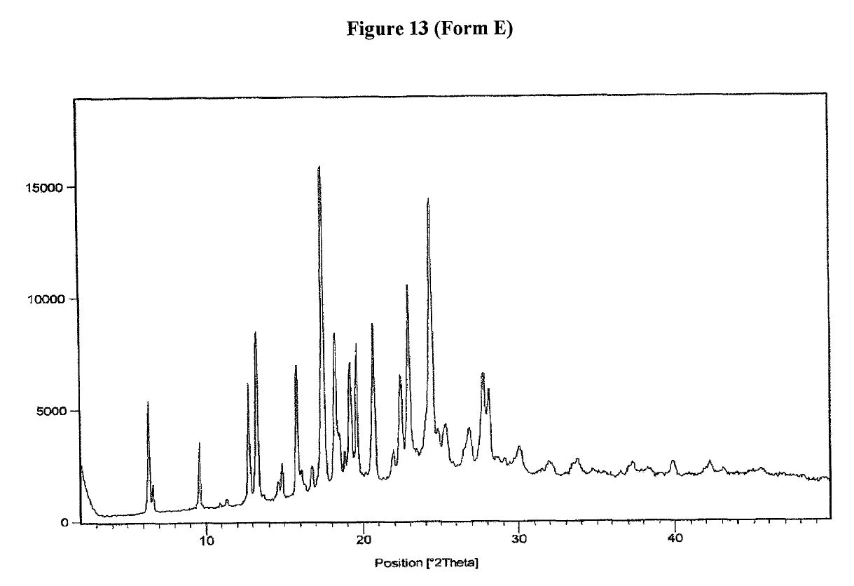

Crystalline carvedilol dihydrogen phosphate Form E is characterized by powder X-ray diffraction pattern as shown in FIG. 13 with peaks at 6.38, 6.63, 9.61, 11.35, 12.79, 13.30, 14.61, 14.87, 15.84, 16.77, 17.52, 18.29, 18.85, 19.25, 19.67, 20.75, 21.97, 22.49, 23.02, 24.47, 25.39, 26.92, 27.78, 28.12, 30.08, 31.92, 33.80, 39.90 and 42.18±0.2 θ values.

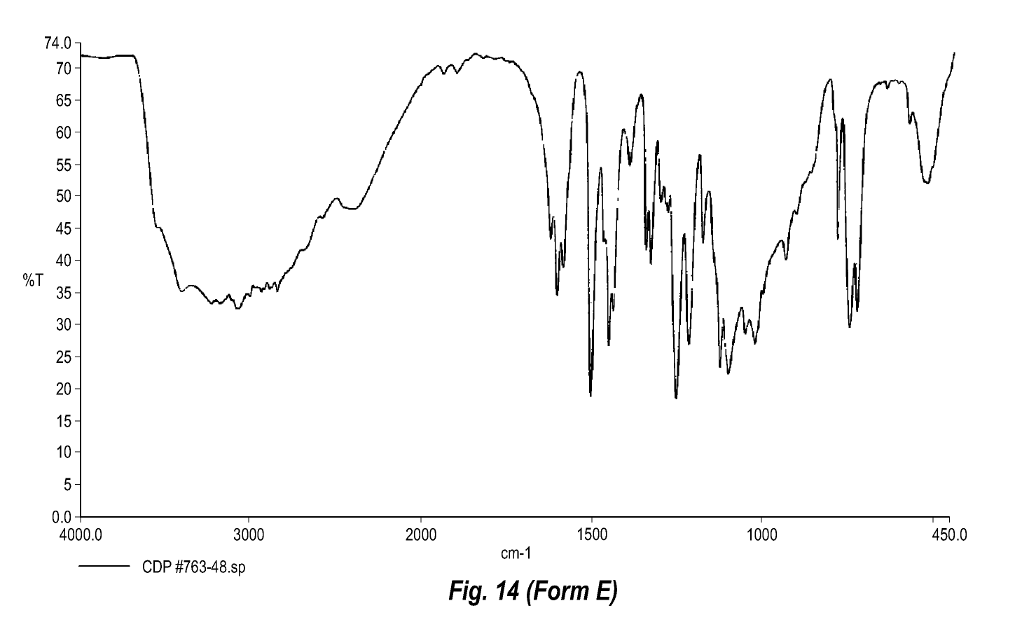

Crystalline carvedilol dihydrogen phosphate Form E is further characterized by IR with absorption bands (cm⁻¹) at 524, 646, 906, 935, 1001, 1051, 2411, 2840, 2884, 3064, and 3223 respectively; as depicted in FIG. 14.

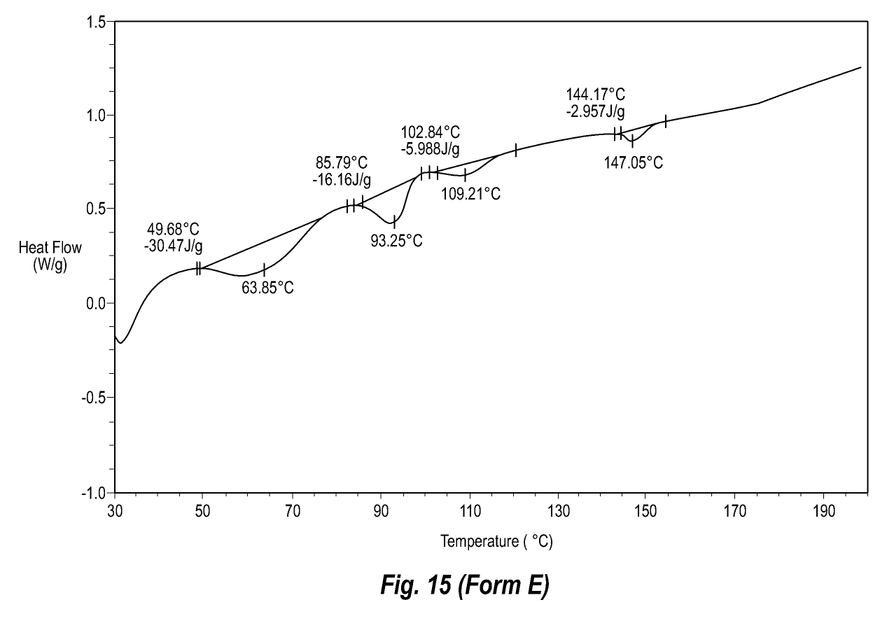

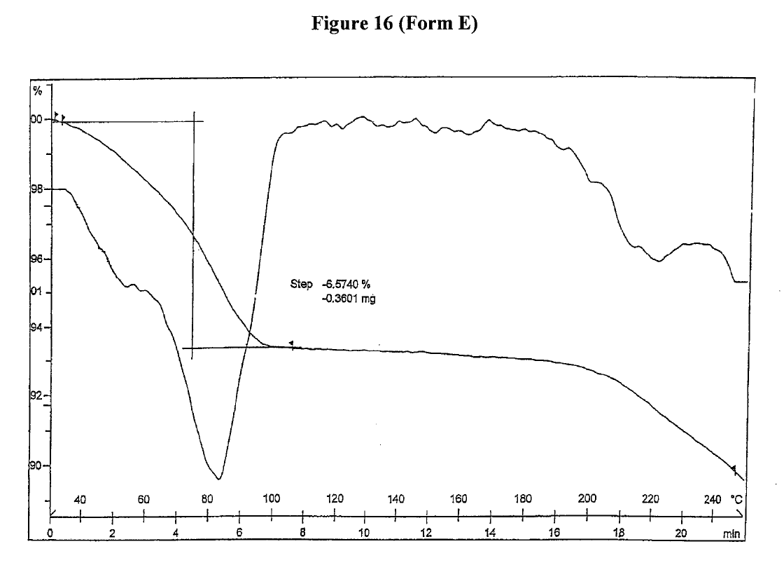

Crystalline carvedilol dihydrogen phosphate Form E is further characterized by the DSC (FIG. 15), which shows three broad endothermic desolvation peaks at 64° C., 93° C., and 109° C. followed by a melting endotherm at 147° C. Crystalline carvedilol dihydrogen phosphate Form E is dihydrate having 5.0-7.0% of water content i.e. 2 moles of water is attached to one mole of carvedilol dihydrogen phosphate, which is analyzed by its TGA data shown in FIG. 16.

The present invention also provides the preparation of crystalline carvedilol dihydrogen phosphate Form E, which comprises slow evaporation of saturated solution of carvedilol dihydrogen phosphate in a solvent at ambient temperature for several days or by lyophilization. The solvent used herein selected from the group consisting of but not limited to water, methanol, acetone, acetonitrile or mixture thereof Carvedilol dihydrogen phosphate used herein selected from the group consisting of but not limited to anhydrous or hydrated form. The crystalline product was isolated by filtration followed by drying, e.g. at room temperature and atmospheric pressure. The resulting dihydrate crystalline form has water content typically ranging from approx. 4.0 to 12.0% by weight, in particular from approx. 5.0-7.0% by weight.

The carvedilol dihydrogen phosphate dihydrate Form E can also be prepared from carvedilol base which comprises contacting carvedilol base with a solvent followed by the treatment with 85% phosphoric acid, optionally adding the second solvent. The reaction mixture is cooled and the solid obtained is filtered and dried under vacuum. The solvent used herein is water whereas second solvent used is methanol, acetonitrile and mixture thereof.

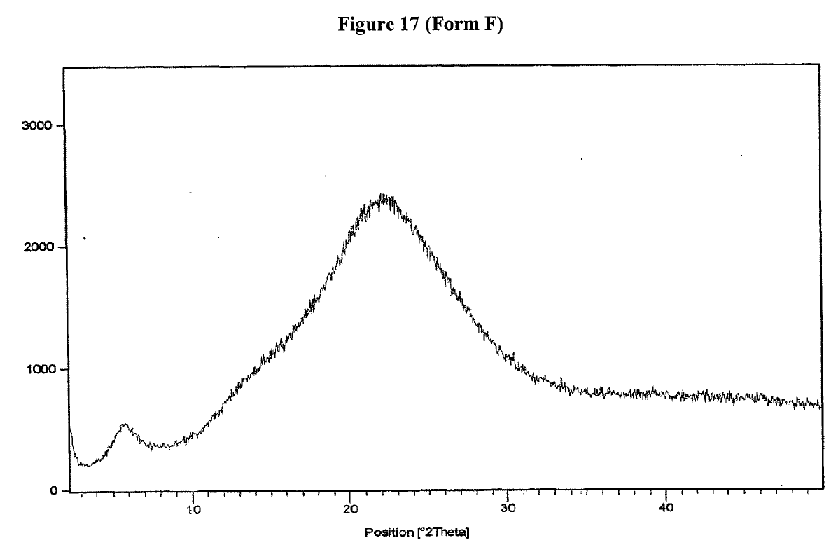

Crystalline carvedilol dihydrogen phosphate Form F is characterized by powder X-ray diffraction pattern as shown in FIG. 17. The amorphous form contains the water up to approximately 1.0 to 4.0% by weight, preferably 1.0 to 2.0% determined by the Karl-Fisher method.

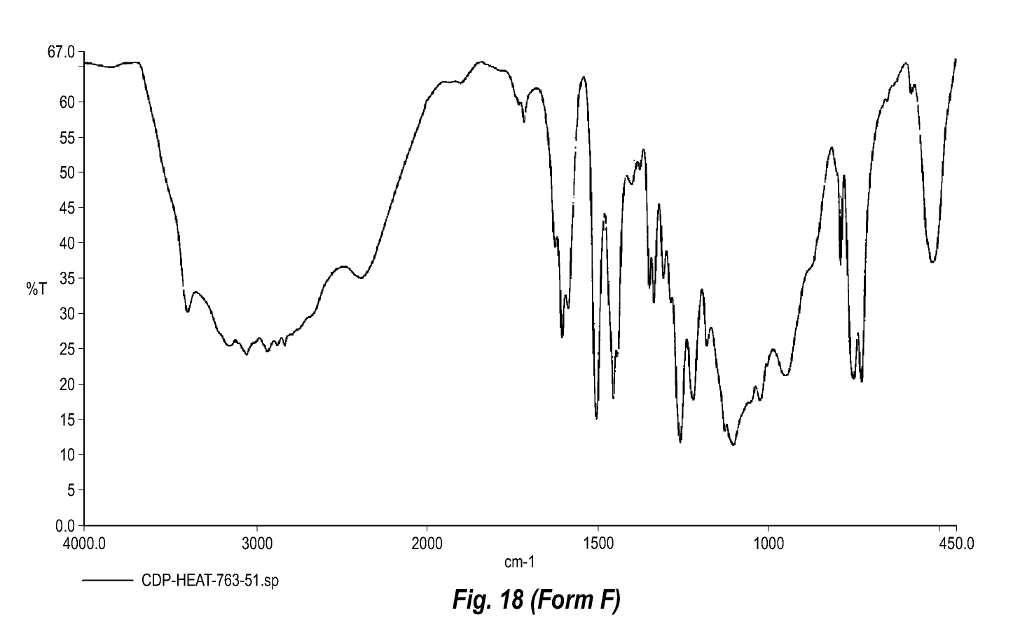

Crystalline carvedilol dihydrogen phosphate Form F is further characterized by IR with absorption bands (cm⁻¹) at 518, 726, 743, 948, 1375, 2383, 2837, 2940, 3060, and 3157 respectively; as depicted in FIG. 18.

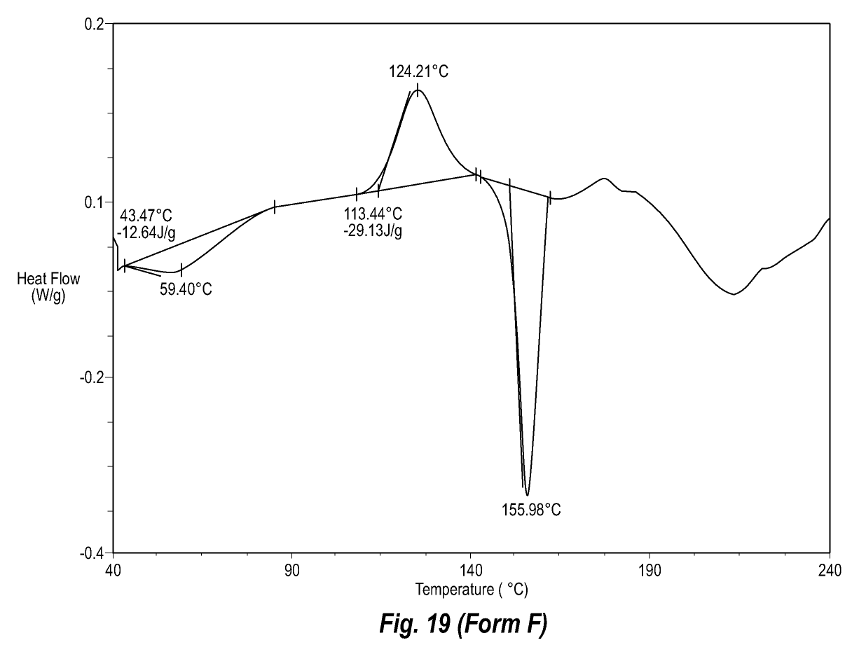

Crystalline carvedilol dihydrogen phosphate Form F is further characterized by the DSC (FIG. 19), which shows the glass transition onset for the amorphous form exactly at 71° C. as measured by modulated DSC. The DSC thermogram of carvedilol dihydrogen phosphate amorphous shows three characteristic peaks; first endothermal peak at an extrapolated onset temperature approx. 45° C. corresponding loss due to the moisture, an exothermic peak at an extrapolated onset temperature ranging from 100 to 140° C., corresponding to the transition from amorphous phase to an crystalline hydrated phase which is identified with a peak at 124° C., and a second endothermal peak at 156° C. (maxima), corresponding to the complete melting of the product.

The present invention also provides the preparation of crystalline carvedilol dihydrogen phosphate Form F, which comprises contacting carvedilol freebase in a solvent selected from the group consisting of alcohol, apolar aprotic, lower aliphatic ketone, ether, hydrocarbon including aliphatic, alicyclic, aromatic, and/or mixtures thereof. The 85% phosphoric acid solution is then added slowly under nitrogen results to the formation of a precipitate, followed by removal of the solvent and recovering the isolated amorphous carvedilol dihydrogen phosphate Form F.

The solvent used is selected from the group consisting of but not limited to acetonitrile, acetone, methanol, ethanol, diethyl ether, isopropyl ether, petroleum ether, hexane, heptane, pentane, cyclohexane, toluene and mixtures thereof. The removal of the solvent may include the processes known in the literature such as distillation, distillation under vacuum, evaporation, spray drying, freeze-drying, filtration, filtration under vacuum, decantation and centrifugation.

The crystalline carvedilol dihydrogen phosphate Form F can also be prepared by spray drying the solution of carvedilol dihydrogen phosphate in a solvent. The solvent used herein selected from the group consisting of but not limited to lower alcohol, ketone, apolar aprotic solvent such as methanol, ethanol, isopropyl alcohol, propanol, butanol, iso-butanol, acetone, dimethylformamide, dimethylsulfoxide, nitromethane, acetonitrile, water and mixture thereof preferably water, methanol, acetone, acetonitrile, propanol, n-butanol and mixture thereof.

Carvedilol dihydrogen phosphate Form F can also be prepared by heating the crystalline carvedilol dihydrogen phosphate dihydrate.

The present invention also provides a process for the preparation of crystalline carvedilol dihydrogen phosphate hemihydrate, which comprises heating any of the crystalline form of carvedilol dihydrogen phosphate selected from the group such as Form A, Form B, Form C or Form D. The carvedilol dihydrogen phosphate hemihydrate can also be prepared by heating the amorphous carvedilol dihydrogen phosphate Form F.

The carvedilol dihydrogen phosphate hemihydrate can also be prepared by contacting the carvedilol dihydrogen phosphate sesquihydrate and/or carvedilol dihydrogen phosphate amorphous in a solvent such as aprotic polar solvents, chlorinated solvents, ethers, esters, lower aliphatic ketones, amides, carbonates or mixtures thereof and recovering. The solvent used herein is selected from the group consisting of but not limited to isopropyl alcohol, methanol, ethyl acetate, dichloromethane, 1,4-dioxane, tetrahydrofuran, nitromethane, diethyl ether, t-butylether, acetone, acetonitrile, dimethylformamide, 2-methoxy ethanol, ethanediol, dimethylcarbonate (DMC), and mixtures thereof. The solid obtained is filtered and dried under vacuum.

The carvedilol dihydrogen phosphate hemihydrate is also be prepared by suspending the carvedilol free base in acetonitrile and adding the 85% phosphoric acid solution in acetonitrile drop-wise and heated the solution at a temperature about 50-70° C., preferably about 60-65° C., to cause the precipitation and recovering the crystalline carvedilol dihydrogen phosphate hemihydrate.

The following non-limiting examples illustrate specific embodiments of the present invention. They are, not intended to be limiting the scope of present invention in any way.

Carvedilol Dihydrogen Phosphate Form A

### EXAMPLE 1

Preparation of Carvedilol Dihydrogen Phosphate Form A from Carvedilol Dihydrogen Phosphate Sesquihydrate

Carvedilol dihydrogen phosphate sesquihydrate (0.5 g) was dissolved in 1,4-dioxane (15 ml) at 75° C. and the undissolved compound was filtered. The filtrate was slowly evaporated at ambient temperature to produce precipitation after 24 hours and the solid was isolated by filtration and dried at 40° C. for 1 hour at atmospheric pressure. PXRD of the wet sample showed it to be Form A.

### EXAMPLE 2

Preparation of Carvedilol Dihydrogen Phosphate Form A from Carvedilol Dihydrogen Phosphate Sesquihydrate

Carvedilol dihydrogen phosphate sesquihydrate (1 g) was suspended in 1,4-dioxane (30 ml) at 50° C. and the mixture was stirred for 1 hour at ambient temperature. The mixture was isolated by filtration and dried at 40° C. for 1 hour at atmospheric pressure.

### EXAMPLE 3

Preparation of Carvedilol Dihydrogen Phosphate Form A from Carvedilol Dihydrogen Phosphate Sesquihydrate

Carvedilol dihydrogen phosphate sesquihydrate (1 g) was suspended in 1,4-dioxane (30 ml) at 75° C. and the mixture was stirred for 1 hour at same temperature. The mixture was isolated by filtration and dried at 40° C. for 1 hour at atmospheric pressure.

### EXAMPLE 4

Preparation of Carvedilol Dihydrogen Phosphate Form A from Carvedilol Dihydrogen Phosphate Sesquihydrate

Carvedilol dihydrogen phosphate sesquihydrate (0.5 g) was suspended in 1,4-dioxane (15 ml) and the resulting slurry was refluxed at 110° C. with stirring for 4 hrs followed by 12 hrs stirring at ambient temperature. The mixture was isolated by filtration and dried at 40° C. for 1 hour under atmospheric pressure.

### EXAMPLE 5

Preparation of Carvedilol Dihydrogen Phosphate Form A from Carvedilol Dihydrogen Phosphate Hemihydrate

Carvedilol dihydrogen phosphate hemihydrate (0.5 g) was suspended in 1,4-dioxane (15 ml) and the resulting slurry was refluxed at 110° C. with stirring for 4 hrs followed by 12 hrs stirring at ambient temperature. The mixture was isolated by filtration and dried at 40° C. for 1 hour under atmospheric pressure.

Carvedilol Dihydrogen Phosphate Form B

### EXAMPLE 6

Preparation of Carvedilol Dihydrogen Phosphate Form B from Carvedilol Dihydrogen Phosphate Sesquihydrate

Carvedilol dihydrogen phosphate sesquihydrate (0.5 g) dissolved in DMF (0.3 ml), followed by the addition of 1,4-dioxane (15 ml) and stirred for 1 hour at ambient temperature. The solid was isolated by filtration and dried at 40° C. under atmospheric pressure. PXRD of the wet sample showed it to be Form B.

### EXAMPLE 7

Preparation of Preparation of Carvedilol Dihydrogen Phosphate Form B from Carvedilol Dihydrogen Phosphate Sesquihydrate

Carvedilol dihydrogen phosphate sesquihydrate (0.3 g) was dissolved in of 1,4-dioxane (7.5 ml) followed by the addition of DMF (1.5 ml) and the solution was allowed for slow evaporation. The solid was isolated by filtration and dried at 40° C. for 1 hour under atmospheric pressure.

Carvedilol Dihydrogen Phosphate Form C

### EXAMPLE 8

Preparation of Carvedilol Dihydrogen Phosphate Form C from Carvedilol Dihydrogen Phosphate Sesquihydrate

Carvedilol dihydrogen phosphate sesquihydrate (0.5 g) was dissolved in 1,2-ethanediol (2.5 ml) at hot condition to form saturated solution which was allowed for crystallization at ambient temperature. The resultant crystals were isolated by filtration and dried at 40° C. for 1.5 hrs under atmospheric pressure. PXRD of the wet sample showed it to be Form C

Carvedilol Dihydrogen Phosphate Form D

### EXAMPLE 9

Preparation of Carvedilol Dihydrogen Phosphate Form D from Carvedilol Dihydrogen Phosphate Sesquihydrate

Carvedilol dihydrogen phosphate sesquihydrate (0.5 g) was dissolved in 1,2-ethanediol (2.5 ml) at hot condition to form saturated solution which was allowed for crystallization at ambient temperature for several days. The precipitate obtained was isolated by filtration and dried at 40° C. for 1.5 hours under atmospheric pressure. PXRD of the wet sample showed it to be Form D

### EXAMPLE 10

Preparation of Carvedilol Dihydrogen Phosphate Form D from Carvedilol Dihydrogen Phosphate Sesquihydrate by Seeding

A saturated solution of carvedilol dihydrogen phosphate sesquihydrate in 1,2-ethanediol (0.5 g in 2.5 ml) was stirred with seeds of Form D obtained from above example 9. A precipitate was obtained in 1 hr and product is isolated by filtration and dried at 40° C. for 1.5 hours under atmospheric pressure.

Carvedilol Dihydrogen Phosphate Dihydrate Form E

### EXAMPLE 11

Preparation of Carvedilol Dihydrogen Phosphate Dihydrate Form E from Carvedilol Base

Carvedilol base (25 g) was dissolved in DM water (150 ml) at ambient temperature and stirred followed by the addition of phosphoric acid solution (7.7 g in 50 ml of water) and maintained at ambient temperature. The solid obtained was isolated by filtration and washed with water followed by drying at room temperature and atmospheric pressure. The water content of solid is measured by Karl-Fisher method as 6.5% w/w. PXRD of wet sample showed it to be CDP dihydrate Form E.

### EXAMPLE 12

Preparation of Carvedilol Dihydrogen Phosphate Dihydrate Form E from Carvedilol Base

Carvedilol base (10 g) was dissolved in DM water (40 ml) at ambient temperature and stirred. Then phosphoric acid solution (3.15 g in 20 ml of water) was added at ambient temperature followed by the addition of methanol (10 ml). The reaction mass was stirred for 2 hours followed by cooling and stirred at the same temperature. The solid obtained was isolated by filtration washed with water and dried at room temperature and atmospheric pressure. PXRD of wet sample showed it to be carvedilol dihydrogen phosphate dihydrate Form E

### EXAMPLE 13

Preparation of Carvedilol Dihydrogen Phosphate Dihydrate Form E from Carvedilol Dihydrogen Phosphate Sesquihydrate

Carvedilol dihydrogen phosphate sesquihydrate (10 g) was taken in a round bottom flask and then added water (200 ml) and acetone (100 ml). The resulting mixture was heated to 80° C. to obtain a clear solution. The solution was concentrated by distilling off the acetone at 48° C. under vacuum (300 mbar). The solution is then cooled to room temperature for 1 h. The resulting solid was filtered and suck dried. The product was identified as carvedilol dihydrogen phosphate dihydrate Form E.

### EXAMPLE 14

Preparation of Carvedilol Dihydrogen Phosphate Dihydrate Form E from Carvedilol Dihydrogen Phosphate Sesquihydrate

Carvedilol dihydrogen phosphate sesquihydrate (2 g) is suspended in methanol (15 ml) and the resulting slurry was stirred for several days at ambient temperature. The resulting solid is identified as carvedilol dihydrogen phosphate dihydrate Form E.

### EXAMPLE 15

Preparation of Carvedilol Dihydrogen Phosphate Dihydrate Form E from Carvedilol Dihydrogen Phosphate Sesquihydrate

Carvedilol dihydrogen phosphate sesquihydrate (2 g) is suspended in methanol (15 ml) and the resulting slurry was stirred for 48 hours at ambient temperature. The resulting solid is identified as carvedilol dihydrogen phosphate dihydrate Form E.

### EXAMPLE 16

Preparation of Carvedilol Dihydrogen Phosphate Dihydrate Form E from Carvedilol Dihydrogen Phosphate Hemihydrate

Carvedilol dihydrogen phosphate hemi-hydrate (2 g) was dissolved in methanol (15 ml) and stirred at ambient temperature. The solid obtained was isolated by filtration and washed with methanol. The resulting solid is identified as carvedilol dihydrogen phosphate dihydrate Form E.

### EXAMPLE 17

Preparation of Carvedilol Dihydrogen Phosphate Dihydrate Form E from Carvedilol Dihydrogen Phosphate Hemihydrate

Carvedilol dihydrogen phosphate hemihydrate (2 g) was dissolved in methanol (15 ml) and the resulting slurry was stirred for several days at ambient temperature. The solid obtained was identified as carvedilol dihydrogen phosphate dihydrate Form E.

### EXAMPLE 18

Preparation of Carvedilol Dihydrogen Phosphate Dihydrate Form E by Lyophilization

Carvedilol dihydrogen phosphate sesquihydrate (2 g) was dissolved in a mixture of water and methanol (75:35 v/v) and the resulting solution was subjected to freeze drying (−104° C. and below 200 Torr vacuum). The obtained solid was identified as carvedilol dihydrogen phosphate dihydrate Form E.

### EXAMPLE 19

Preparation of Carvedilol Dihydrogen Phosphate Dihydrate Form E by Lyophilization

Carvedilol dihydrogen phosphate sesquihydrate (2 g) was dissolved in 40% methanol in water and the resulting solution was subjected to freeze-drying (−104° C. and below 200 Torr vacuum). The obtained solid was identified as carvedilol dihydrogen phosphate dihydrate Form E.

### EXAMPLE 20

Preparation of Carvedilol Dihydrogen Phosphate Dihydrate Form by Lyophilization

Carvedilol dihydrogen phosphate sesquihydrate (4 g) was dissolved in a mixture of water and methanol (v/v 1:1) and the resulting solution was subjected to freeze-drying (−104° C. and below 200 Torr vacuum). The obtained solid is identified as carvedilol dihydrogen phosphate dihydrate Form E.

Amorphous Carvedilol Dihydrogen Phosphate Form F

### EXAMPLE 21

Preparation of Amorphous Carvedilol Dihydrogen Phosphate Form F from Carvedilol Dihydrogen Phosphate Dihydrate

Carvedilol dihydrogen phosphate dihydrate (2 g), obtained as described in examples was placed in a covered petri-dish and heated in a static dryer under vacuum at 75° C. for several days, preferably 4-9 days, more preferably 2-3 days. The amorphous form can contain water up to approximately 1 to 4% by weight, preferably 1 to 2% by weight. PXRD of the sample showed it to be CDP amorphous Form F.

### EXAMPLE 22

Preparation of Carvedilol Dihydrogen Phosphate Amorphous Form F from Carvedilol Base

Carvedilol base (2 g, 1 eq.) was suspended in acetonitrile (76 ml) and the resulting slurry was heated for complete dissolution. The solution was cooled to room temperature and stirred at this temperature. Then, 85% phosphoric acid (0.63 g, 1.1 eq.) in acetonitrile (5 ml) was added to the solution slowly. A white gummy suspension was formed which upon complete distillation of the solvent under vacuum at 50° C. leaving white foam. Additional drying under reduced pressure for 1 hr results free solid product (2 g, 98%) of amorphous carvedilol dihydrogen phosphate Form F. PXRD of the obtained solid showed it to be amorphous Form F.

### EXAMPLE 23

Preparation of Carvedilol Dihydrogen Phosphate Amorphous Form F from Carvedilol Base

Carvedilol base (2 g, 1 eq.) was suspended acetonitrile (76 ml) and the resulting slurry was heated to 45° C. for complete dissolution. The solution was cooled to room temperature and stirred at this temperature for 15 min. Then, 85% phosphoric acid (0.63 g, 1.1 eq.) in 10 ml of acetonitrile was added to the solution slowly for a period of 0.5 hr. A white gummy suspension was formed which upon complete distillation of the solvent under vacuum at 50° C. leaving white foam. Additional drying under reduced pressure for 1 hr results free solid product (2 g, yield 98%) of amorphous carvedilol dihydrogen phosphate Form F. PXRD of the sample showed it to be amorphous Form F.

### EXAMPLE 24

Preparation of Carvedilol Dihydrogen Phosphate Amorphous Form F from Carvedilol Base

Carvedilol base (10 g, 1 eq.) was suspended in acetonitrile (376 ml, 38 vol) and the resulting slurry was heated for complete dissolution. The solution was cooled to room temperature and stirred at this temperature. Then, 85% phosphoric acid (3.15 g, 1.1 eq.) in acetonitrile (50 ml) was added to the solution slowly. A white gummy suspension was formed which upon complete distillation of the solvent under vacuum at 50° C. leaving white foam. Additional drying under reduced pressure for 1 hr results a free solid which was stirred in isopropyl ether (50 ml). The solid product obtained was filtered and dried under vacuum at 40° C. for 32 hrs. The product was identified to be amorphous carvedilol dihydrogen phosphate Form F. Its water content is approx. 3.1% w/w. PXRD of the sample showed it to be amorphous Form F.

### EXAMPLE 25

Preparation of Carvedilol Dihydrogen Phosphate Amorphous Form F from Carvedilol Base

Carvedilol base (10 g, 1 eq.) was suspended acetonitrile (376 ml, 38 vol) and the resulting slurry was heated for complete dissolution. The solution was cooled to room temperature and stirred at this temperature. Then, 85% aqueous phosphoric acid (0.63 g, 1.1 eq.) in acetonitrile (50 ml) was added to the solution. A white gummy suspension was formed which upon complete distillation of the solvent under vacuum at 50° C. leaving white foam. Additional drying under reduced pressure for 1 hr results a free solid which on stirring with heptane (50 ml) gave solid product. The obtained solid was filtered and dried under vacuum at 40° C. for 3 hr. The product was identified to be amorphous Form F. Its water content is approx. 3.6% w/w. PXRD of the sample showed it to be amorphous Form F.

### EXAMPLE 26

Preparation of Carvedilol Dihydrogen Phosphate Amorphous Form F from Carvedilol Base

Carvedilol base (2 g, 1 eq.) was suspended in acetonitrile (76 ml, 38 vol) and the resulting slurry was heated for complete dissolution. The solution was cooled to room temperature and stirred at this temperature. Then, 85% phosphoric acid (0.63 g, 1.1 eq.) in acetonitrile (20 ml) was added to the solution slowly. A white gummy suspension was formed which upon complete distillation of the solvent under vacuum at 50° C. leaving white foam. Additional drying under reduced pressure for 1 hr results a free solid. Then add pentane (5 vol) and stir. The solid product obtained was filtered and dried under vacuum at 40° C. for 3 hr. The product was identified to be amorphous carvedilol dihydrogen phosphate From F. PXRD of the sample showed it to be carvedilol dihydrogen phosphate amorphous Form F.

### EXAMPLE 27

Preparation of Carvedilol Dihydrogen Phosphate Amorphous Form F from Carvedilol Base

Carvedilol base (2 g, 1 eq.) was suspended in acetonitrile (76 ml) and the resulting slurry was heated for complete dissolution. The solution was cooled to room temperature and stirred at this temperature. Then, 85% phosphoric acid (0.63 g, 1.1 eq.) in acetonitrile (10 ml) was added to the solution slowly. A white gummy suspension was formed which upon complete distillation of the solvent under vacuum at 50° C. leaving white foam. Additional drying under reduced pressure for 1 hr results a free solid. Then diethyl ether (5 vol) was added and stirred. The solid product obtained was filtered and dried under vacuum at 40° C. for 3 hr. The product was identified to be amorphous carvedilol dihydrogen phosphate Form F. PXRD of the sample showed it to be carvedilol dihydrogen phosphate amorphous form.

### EXAMPLE 28

Preparation of Carvedilol Dihydrogen Phosphate Amorphous Form F from Carvedilol Base

Carvedilol base (2 g, 1 eq.) was suspended acetonitrile (76 ml) and the resulting slurry was heated to 45° C. for complete dissolution. The solution was cooled to room temperature and stirred at this temperature. Then, 85% phosphoric acid (0.63 g, 1.1 eq.) in acetonitrile (10 ml) was slowly added to the solution. A white gummy suspension was formed which upon complete distillation of the solvent under vacuum at 50° C. leaving white foam. Additional drying under reduced pressure for 1 hr results a free solid. Then cyclohexane (5 vol) was added and stirred. The solid product obtained was filtered and dried under vacuum at 40° C. for 3 hr. PXRD of the sample showed it to be amorphous Form F.

### EXAMPLE 29

Preparation of Carvedilol Dihydrogen Phosphate Amorphous Form F from Carvedilol Base

Carvedilol base (2 g, 1 eq.) was suspended acetonitrile (76 ml) and the resulting slurry was heated for complete dissolution. The solution was cooled to room temperature and stirred at this temperature. Then, 85% phosphoric acid (0.63 g, 1.1 eq.) in acetonitrile (20 ml) was slowly added to the solution. A white gummy suspension was formed which upon complete distillation of the solvent under vacuum at 50° C. leaving white foam. Additional drying under reduced pressure for 1 hr results a free solid. Then hexane (5 vol) was added and stirred. The solid product was obtained was filtered and dried under vacuum at 40° C. for 3 hr. The product was identified to be amorphous.

### EXAMPLE 30

Preparation of Carvedilol Dihydrogen Phosphate Amorphous Form F from Carvedilol Base

Carvedilol base (2 g, 1 eq.) was dissolved in Methanol (76 ml) and the resulting solution and stirred. Then, 85% phosphoric acid (0.63 g, 1.1 eq.) in methanol (10 ml) was slowly added to the solution. The solution was completely distilled off under vacuum at 50° C. leaving white foam. Additional drying under reduced pressure for 1 hr results free solid as an amorphous product Form F (2 g, 98%).

### EXAMPLE 31

Preparation of Carvedilol Dihydrogen Phosphate Amorphous Form F from Carvedilol Base

Carvedilol base (2 g, 1 eq.) was dissolved in acetone (76 ml) and stirred. Then 85% phosphoric acid (0.63 g, 1.1 eq.) in acetone (10 ml) was slowly added to the solution. The solution was completely distilled off under vacuum at 50° C. leaving white foam. Additional drying under reduced pressure for 1 hr results free solid as an amorphous product Form F (2 g, yield 98%).

### EXAMPLE 32

Preparation of Carvedilol Dihydrogen Phosphate Amorphous Form F from Carvedilol Base

Carvedilol base (2 g, 1 eq.) was dissolved in methanol (76 ml) and stirred. Then 85% phosphoric acid (0.63 g, 1.1 eq.) in methanol (10 ml) was slowly added to the solution. The solution was completely distilled off under vacuum at 50° C. leaving white foam. Additional drying under reduced pressure for 1 hr results free solid. The solid obtained was stirred in isopropyl ether (10 ml) to get amorphous product Form F, which was filtered and dried under vacuum at 40° C.

Carvedilol Dihydrogen Phosphate Hemihydrate

### EXAMPLE 33

Carvedilol dihydrogen phosphate sesquihydrate (1 g) was dissolved in acetonitrile (5 ml) and methanol (1 ml) mixture at 80° C., cooling the solution and allowed for crystallization at ambient temperature for overnight. The resulting solid precipitated was identified as carvedilol dihydrogen phosphate hemihydrate.

### EXAMPLE 34

Carvedilol dihydrogen phosphate sesquihydrate (1 g) was suspended in dimethylcarbonate (8 ml) and the resulting slurry was stirred at room temperature for overnight. The solid obtained was filtered and dried at ambient temperature. The obtained solid was identified as carvedilol dihydrogen phosphate hemihydrate.

### EXAMPLE 35

Amorphous carvedilol dihydrogen phosphate (1 g) was dissolved in methanol (5 ml) and resulting slurry was stirred at room temperature for 1 hour. The solid obtained was filtered and dried at ambient temperature. The obtained solid was identified as carvedilol dihydrogen phosphate hemihydrate.

### EXAMPLE 36

Amorphous carvedilol dihydrogen phosphate (1 g) was heated in a static dryer above 100° C. for 72-84 hrs. The obtained solid was identified as carvedilol dihydrogen phosphate hemihydrate.

### EXAMPLE 37

Carvedilol base (2 g, 1 eq.) was suspended acetonitrile (76 ml) and the resulting slurry was heated for complete dissolution. The solution was cooled to room temperature and stirred at this temperature. Then, 85% phosphoric acid (0.63 g, 1.1 eq.) in acetonitrile (20 ml) was slowly added to the solution. The reaction mixture was heated to 60° C. the solid obtained was filtered and isolated by filtration. The obtained solid was identified as carvedilol dihydrogen phosphate hemihydrate.

Certain modifications and improvements of the disclosed invention will occur to those skilled in the art without departing from the scope of invention, which is limited only by the appended claims.

1. A carvedilol dihydrogen phosphate Form A.

2. The crystalline carvedilol dihydrogen phosphate Form A according to claim 1, wherein said form is characterized by having an X-ray powder diffraction pattern peaks at 4.5, 7.5, 8.7, 14.9, 15.3, 15.6, 19.3, 22.3, 22.9, 23.3, 23.7, 26.2, 28.6 and 30.1±0.2 2θ values.

3. (canceled)

4. (canceled)

5. The crystalline carvedilol dihydrogen phosphate Form A according to claim 1, wherein said form is characterized by differential scanning calorimetry (DSC) as depicted in FIG. 2 and thermo gravimetric analysis (TGA) as depicted in FIG. 3.

6. The crystalline carvedilol dihydrogen phosphate Form A according to claim 1, wherein said crystalline Form A is a solvate containing 6-13% of dioxane.

7. A process for preparing crystalline carvedilol dihydrogen phosphate Form A as claimed in claim 1, the process comprising:
(a) contacting carvedilol dihydrogen phosphate in 1,4-dioxane; and
(b) isolating the solid obtained and drying the resultant.

8. The process according to claim 7, wherein the carvedilol dihydrogen phosphate used is anhydrous or hydrated form.

9. The process according to claim 7, wherein the carvedilol dihydrogen phosphate is contacted with 1,4-dioxane at ambient temperature to reflux temperature.

10. A crystalline carvedilol dihydrogen phosphate Form B.

11. The crystalline carvedilol dihydrogen phosphate Form B according to claim 10, wherein said form is characterized by having an X-ray powder diffraction pattern peaks at 8.0, 9.2, 13.3, 14.0, 16.2, 17.7, 21.7, 26.8 and 27.5±0.2 2θ values.

12. (canceled)

13. (canceled)

14. The crystalline carvedilol dihydrogen phosphate Form B according to claim 10, wherein said form is characterized by differential scanning calorimetry (DSC) as depicted in FIG. 5 and thermo gravimetric analysis (TGA) as depicted in FIG. 6.

15. A process for preparing crystalline carvedilol dihydrogen phosphate Form B as claimed in claim 10, the process comprising:
(a) contacting carvedilol dihydrogen phosphate in a mixture of dioxane and dimethylformamide; and
(b) isolating the solid obtained and drying the resultant.

16. The process according to claim 15, wherein the carvedilol dihydrogen phosphate used is anhydrous or hydrated form.

17. A crystalline carvedilol dihydrogen phosphate Form C.

18. The crystalline carvedilol dihydrogen phosphate Form C according to claim 17, wherein said form is characterized by having an X-ray powder diffraction pattern characterized by peaks at 6.6, 8.9, 13.1, 13.6, 15.4, 16.3, 17.5, 17.7, 18.3, 18.7, 19.3, 19.7, 21.7, 22.8, 23.7, 24.8, 26.7, 29.4, 30.0, 32.9 and 37.3±0.2 2θ values.

19. (canceled)

20. (canceled)

21. The crystalline carvedilol dihydrogen phosphate Form C according to claim 17, wherein said form is characterized by differential scanning calorimetry (DSC) as depicted in FIG. 8 and thermo gravimetric analysis (TGA) as depicted in FIG. 9.

22. The crystalline carvedilol dihydrogen phosphate Form C according to claim 17, wherein said crystalline Form C is a solvate containing 2-6% of ethanediol.

23. A process for preparing crystalline carvedilol dihydrogen phosphate Form C as claimed in claim 17, the process comprising:
(a) contacting carvedilol dihydrogen phosphate in ethanediol;
(b) evaporating the ethanediol; and
(c) isolating the solid obtained and drying the resultant.

24. The process according to claim 23, wherein the evaporation is done at slow rate.

25. The process according to claim 23, wherein the carvedilol dihydrogen phosphate used is anhydrous or hydrated form.

26. A crystalline carvedilol dihydrogen phosphate Form D.

27. The crystalline carvedilol dihydrogen phosphate Form D according to claim 26, wherein said form is characterized by having an X-ray powder diffraction pattern peaks at 4.5, 7.8, 8.6, 8.8, 9.0, 12.4, 15.5, 18.7, 20.2, 22.1, 25.8, 28.0, 34.1, 34.7, 38.3, and 47.5±0.2 2θ values.

28. (canceled)

29. (canceled)

30. The crystalline carvedilol dihydrogen phosphate Form D according to claim 26, wherein said form is characterized by differential scanning calorimetry (DSC) as depicted in FIG. 11 and thermo gravimetric analysis (TGA) as depicted in FIG. 12.

31. The crystalline carvedilol dihydrogen phosphate Form D according to claim 26, wherein said crystalline Form D is a solvate containing 6-11% of ethanediol.

32. A process for preparing crystalline carvedilol dihydrogen phosphate Form D as claimed in claim 26, the process comprising:
(a) preparing the saturated solution of carvedilol dihydrogen phosphate in a ethanediol;
(b) optionally seeding with Form D crystals;
(c) evaporating the solution; and
(d) isolating the solid obtained and drying the resultant.

33. The process according to claim 32, wherein the evaporation is done at slow rate.

34. The process according to claim 32, wherein the carvedilol dihydrogen phosphate used is anhydrous or hydrated form.

35. A crystalline carvedilol dihydrogen phosphate Form E.

36. The crystalline carvedilol dihydrogen phosphate Form E according to claim 35, wherein said form is characterized by having an X-ray powder diffraction pattern peaks at 12.7, 13.3, 14.8, 16.7, 17.5, 19.2, 19.6, 23.0, 27.7 and 28.1±0.2 2θ values.

37. (canceled)

38. (canceled)

39. The crystalline carvedilol dihydrogen phosphate Form E according to claim 35, wherein said form is characterized by infrared spectrum (IR) as depicted in FIG. 14.

40. The crystalline carvedilol dihydrogen phosphate Form E according to claim 35 characterized by differential scanning calorimetry (DSC) as depicted in FIG. 15 and thermo gravimetric analysis (TGA) as depicted in FIG. 16.

41. The crystalline carvedilol dihydrogen phosphate Form E according to claim 35, wherein said form is dihydrate.

42. A process for preparing crystalline carvedilol dihydrogen phosphate Form E as claimed in claim 35, the process comprising:
(a) preparing the saturated solution of carvedilol dihydrogen phosphate in a solvent;
(b) evaporating the solution; and
(c) isolating the solid obtained and drying the resultant.

43. The process according to claim 42, wherein the carvedilol dihydrogen phosphate used is anhydrous or hydrated form.

44. The process according to claim 42, wherein the solvent used is water, methanol, acetone, acetonitrile or mixture thereof.

45. The process according to claim 42, wherein the evaporation is done at slow rate.

46. A process for preparing crystalline carvedilol dihydrogen phosphate Form E as claimed in claim 35, the process comprises lyophilization of the carvedilol dihydrogen phosphate in a solvent.

47. The process according to claim 46, wherein the solvent used is water, methanol, acetone, acetonitrile or mixture thereof.

48. The process according to claim 46, wherein the carvedilol dihydrogen phosphate used is anhydrous or hydrated form.

49. A process for preparing crystalline carvedilol dihydrogen phosphate Form E as claimed in claim 35, the process comprising:
(a) contacting carvedilol base with a solvent;
(b) treated with 85% phosphoric acid;
(c) optionally adding the second solvent;
(d) cooling the reaction mixture; and
(e) filtering the solid obtained and dried.

50. The process according to claim 49, wherein the solvent is water and second solvent is methanol or acetonitrile or mixture thereof.

51. An amorphous carvedilol dihydrogen phosphate Form F.

52. The amorphous carvedilol dihydrogen phosphate Form F according to claim 51, wherein said form is characterized by having a X-ray powder diffraction pattern as depicted in FIG. 17.

53. The amorphous carvedilol dihydrogen phosphate Form F according to claim 51, wherein said form is characterized by having infrared spectrum (IR) as depicted in FIG. 18.

54. The amporphus carvedilol dihydrogen phosphate Form F according to claim 51, wherein said form is characterized by differential scanning calorimetry (DSC) as depicted in FIG. 19.

55. A process for preparing amorphous carvedilol dihydrogen phosphate Form F as claimed in claim 51, the process comprising:
(a) contacting carvedilol free base with a solvent;
(b) treating with 85% phosphoric acid; and
(c) isolating the solid obtained and dried.

56. The process according to claim 55, wherein the solvent is selected from a group consisting of alcohol, apolar aprotic, lower aliphatic ketone, ether, hydrocarbon including aliphatic, alicyclic, aromaticor mixtures thereof.

57. (canceled)

58. The process according to claim 55, wherein the isolation is done by removal of the solvent using distillation, distillation under vacuum, evaporation, spray drying, freeze-drying, filtration, filtration under vacuum, decantation and centrifugation.

59. A process for preparing carvedilol dihydrogen phosphate Form F as claimed in claim 51, the process comprising heating a crystalline carvedilol dihydrogen phosphate dihydrate.

60. A process for preparing carvedilol dihydrogen phosphate Form F as claimed in claim 51, the process comprising:
(a) dissolving carvedilol dihydrogen phosphate in a solvent;
(b) isolating the solid obtained and dried.

61. The process according to claim 60, wherein the solvent is selected from a group consisting of lower alcohol, ketone, apolar aprotic solvent or mixture thereof.

62. (canceled)

63. The process according to claim 61, wherein the solvent is water, methanol, acetone, acetonitrile, propanol, n-butanol or mixture thereof.

64. The process according to claim 60, wherein the isolation is done by spray drying.

65. The process according to claim 60, wherein the carvedilol dihydrogen phosphate used is anhydrous or hydrated form.

66. A process for preparing crystalline carvedilol dihydrogen phosphate hemihydrate, the process comprises the heating of carvedilol dihydrogen phosphate.

67. The process according to claim 66, wherein the carvedilol dihydrogen phosphate is crystalline Form A, Form B, Form C, Form D or amorphous Form F.

68. A process for preparing crystalline carvedilol dihydrogen phosphate hemihydrates, the process comprising:
(a) contacting the carvedilol dihydrogen phosphate in a solvent;
(b) isolating the solid obtained and dried.

69. The process according to claim 68, wherein the solvent is selected from the group comprising aprotic polar solvent, chlorinated solvent, ether, ester, lower aliphatic ketone, amide, carbonate or mixtures thereof.

70. (canceled)

71. The process according to claim 68, wherein the carvedilol dihydrogen phosphate used as sesquihydrate and/or as amorphous.

72. A process for preparing carvedilol dihydrogen phosphate hemihydrate, the process comprising:
(a) contacting carvedilol free base in acetonitrile;
(b) adding 85% phosphoric acid solution in acetonitrile;
(c) heating the solution; and
(d) recovering the crystalline carvedilol dihydrogen phosphate hemihydrate.

73. The process according to claim 72, wherein heating is done at a temperature about 50-70° C.

74. (canceled)
